(19) United States
(12) Patent Application Publication
(10) Pub. No.: US 2015/0157375 A1
(43) Pub. Date: Jun. 11, 2015

(54) TENSIONING ELEMENT FOR FIXING THE FRACTURE ENDS OF THE BONES IN A BONE FRACTURE

(71) Applicant: Hipp medical AG, Kolbingen (DE)

(72) Inventor: Markus Waizenegger, Kolbingen (DE)

(21) Appl. No.: 14/407,237

(22) PCT Filed: Mar. 13, 2013
(86) PCT No.: PCT/EP2013/000749

§ 371 Date: Dec. 11, 2014

(30) Foreign Application Priority Data
Jun. 13, 2012 (DE) 102012105125.9

(51) Int. Cl. A61B 17/82; A61B 17/68; A61B 17/80

(52) U.S. Cl. CPC A61B 17/82; A61B 17/8085; A61B 17/8004; A61B 17/68; A61B 2017/681

(57) ABSTRACT

The invention proposes a tensioning element (1) for fixing the fracture ends of the bones in a bone fracture with a contoured body which is hollow when viewed from above and comprises a circumferential wall (6). The contoured body comprises two face sided receiving sections (a, 8b) lying opposite to each other, for receiving fastening means that can be pushed through the contoured body, and two lateral flanks with angled sections (10). The circumferential wall (6) of the contoured body is, at least in sections, in the region of the face side receiving sections (8a, 8b) and the lateral angled sections (10) resiliently deformable. Further, a defined elastic behavior of the tensioning element (1) is impressed via the angled sections (10) in such a way that a predetermined tension can be generated between the receiving sections (a, 8b). According to the present invention, the contoured body comprises at least one articulation (2, 4) for compensating and distributing stress peaks, wherein the at least one articulation (2, 4) is present in the region of the circumferential wall (6).

The invention relates to a tensioning element for fixing the fracture ends of the bones in a bone fracture according to claim 1.

When a bone fracture is immobilized insufficiently, a callus formation, i.e. a tyloma-like thickening of the ends of the fracture from overgrowing bone tissue, can occur. In order to avoid such an indirect fracture healing via a callus, bone plates are used which are applied and attached to the outer surface of a broken bone so that the fracture site is set, i.e. fixed, during the healing process.

In addition, the healing process of a fracture is favorably influenced when a compression is applied on the jointed site of fracture. In this way, a particularly close adaptation is caused, i.e. a low clearance by which the broken ends grow to each other again.

From DE 20 2011 109808 U1 is known a tensioning element which is composed of a contoured body designed as a circumferential wall with resilient angled sections and receiving sections. The positioning of the tension element takes places by the receiving sections, in which cortical screws can be inserted and placed into the bone. In order to achieve the desired compression of the fracture ends, prior to the positioning, and fixation respectively, of the tensioning element to the bone, from the outside a pressure pointing inwardly is applied to the angled sections. It reveals as a disadvantage of this tensioning element that in particular in the angled portions mechanical maximum stresses occur which can result in a material failure or in a potential non-uniformity of the tensioning element.

Thus, it is the underlying object of the present invention to provide a tensioning element which is suitable to uniformly distribute the mechanical maximum stresses within the contoured body.

This object is solved by a tensioning element with the features of claim 1.

According to the invention, a tensioning element for fixing the fracture ends of the bones in a bone fracture with a contoured body which is hollow when viewed from above and comprises a circumferential wall is proposed. The contoured body comprises two face sided receiving sections lying opposite to each other, for receiving fastening means that can be pushed through the contoured body, and two lateral flanks with angled sections. The circumferential wall of the contoured body is, at least in sections, in the region of the face side receiving sections and the lateral angled sections resiliently deformable. Further, a defined elastic behavior of the tensioning element is impressed via the angled sections in such a way that a predetermined tension can be generated between the receiving sections. According to the present invention, the contoured body comprises at least one articulation for compensating and distributing stress peaks, wherein the at least one articulation is present in the region of the circumferential wall.

By forming an articulation within the circumferential wall, the occurring maximum stresses can get very well branched and distributed in the tensioning element and thus compensated. By this, a more homogeneous stress distribution in the tensioning element can be achieved, whereby weak points can be avoided. A material wear occurring by the maximum stresses is particularly well counteracted.

The formed articulation alters in a particular advantageous manner the original force and stress curve, meaning the curve without the articulation, to regions with lower stresses, whereby the tensioning element is uniformly loaded and even relieved in some areas.

According to the invention, with a tensioning element provided with articulations it can be prevented that the tensioning element breaks in the region of the maximum stresses occurring otherwise and thus fails during the healing of the bone fracture due to excessive material load.

Moreover, the articulations can be adapted individually to the forces needed for compression and the resulting stresses in dependency of the size of the tensioning element and the bone fracture to be cured, for example comminuted fracture.

A further advantage can be seen in that the risk of breakage of the tensioning element when accidentally overloaded, for example when fixing the tensioning element at the fracture ends of the bones, is reduced. Accordingly, a potential breakage of the tensioning element can be avoided at all and/or material fatigue of the tensioning element is prevented.

Further embodiments of the tensioning element of the present invention are subject-matter of dependent claims 2 to 9.

The articulation of the tensioning element of the present invention can be deformable. By the deformation of the articulation the stresses, in particular maximum stresses, can be distributed advantageously in the tensioning element and can be compensated. The articulation here can deform either elastic or plastic, depending on the received maximum stresses. Since the articulations are formed in regions where neither the positioning, nor the desired compression, are disturbed, the deformable articulation does not affect the tensioning element and its functions in a negative way.

In an alternative way, the articulation can also be rotatable. By configuring a rotatable articulation the maximum stresses are effectively absorbed and afterwards distributed. The rotatable formation of the articulation impairs neither the desired compression of the fracture ends, nor the positioning of the tensioning element.

Further, the articulation can be configured in such a way that it is positioned at docking points of the angled sections at the receiving sections. In particular in this area the maximum stresses favorably get branched and thus compensated because there the stresses merge into each other and add up.

Moreover, the articulation of the tensioning element of the present invention can be integrated in the lateral flanks. Because prior to the positioning of the tensioning element the same gets compressed in order to achieve a longitudinal compression of the fracture ends in the following, high stresses occur in particular in the lateral flanks. This increased stresses therefore can be especially well distributed and compensated by forming articulations within the lateral flanks.

Furthermore, at two wall sections opposing each other there can be formed restriction bars facing each other, whose ends contacting each other when the tensioning element is deformed. The restriction of a deformation pathway introduced due to external forces provides protection against deformations which exceed the elastic range of the tensioning element and would result in a permanent alteration of the initial dimensions of the tensioning elements. Moreover, the limit stop can be used as indicator of a predetermined prestressing of the tensioning element.

The articulation of the tensioning element of the present invention can be a hinge. The integration of hinges in the circumferential wall can be made particular easily. A formation of a hinge in the circumferential wall, however, does not mean that the wall is interrupted. The term hinge here means a piano hinge, spring hinge, torque hinge, locking hinge, foil hinge, film hinge or the same.

The articulation of the tensioning element of the present invention, on the other hand, can be a ball joint. In particular in the case of comminuted fractures the ball joint enables a particularly good stress distribution. Also in the case of a curvature of the tensioning element with respect to the longitudinal axis the ball joint can distribute the maximum stresses in a preferred manner and reduce same.

As an alternative, the articulation of the tensioning element of the present invention can be a node joint. The node joint distributes the stresses occurring at its node, in particular the maximum stresses, very well and thus relieves the tensioning element itself.

The features and functions of the present invention described above as well as the further aspects and features will be described in the following by a detailed description of preferred embodiments under reference to the attached figures. In this connection shows:

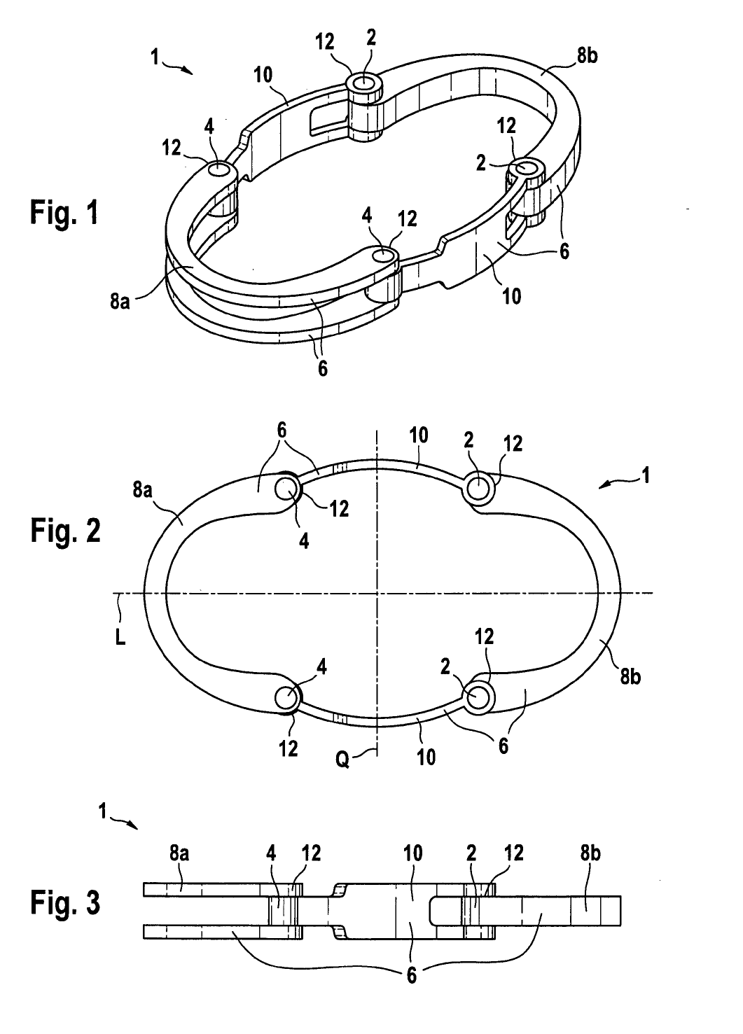

FIG. 1 a perspective view of a first embodiment of a tensioning element of the present invention with hinges;

FIG. 2 a top view of the first embodiment of the tensioning element of the present invention with hinges according to FIG. 1;

FIG. 3 a side view of the first embodiment of the tensioning element of the present invention with hinges according to FIG. 1;

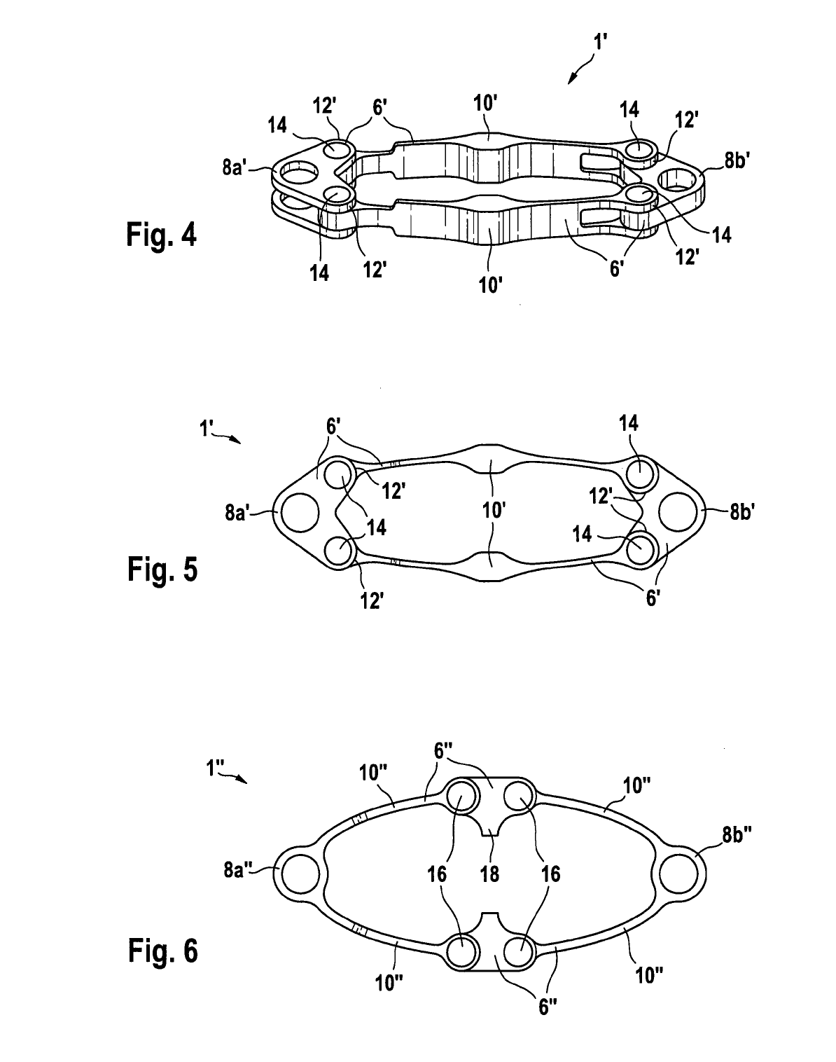

FIG. 4 a perspective view of a second embodiment of a tensioning element of the present invention with node joints;

FIG. 5 a top view of the second embodiment of the tensioning element of the present invention with node joints according to FIG. 4; and

FIG. 6 a top view of a third embodiment of a tensioning element of the present invention with node joints.

FIG. 1 is a perspective view, FIG. 2 is a top view and FIG. 3 is a side view of a first embodiment of a tensioning element 1 of the present invention with hinges 2, 4.

The tensioning element is composed of a contoured body which is hollow when viewed from above and which comprises a circumferential wall 6. The contoured body comprises two receiving sections 8a, 8b which lie opposite to each other with respect to the lateral axis Q of the tensioning element 1 and which are curved outward. Further, the contoured body comprises two lateral flanks with angled sections 10 which lie opposite to each other with respect to the longitudinal axis L of the tensioning element 1 and which are also curved outward. The tensioning element 1 is configured symmetrically with respect to its longitudinal axis L.

As particularly visible in FIG. 3, the corresponding tongue-and-groove structure of the receiving sections 8a, 8b allow that several tensioning elements 1 are connected to each other. For example, the receiving section 8b of the tensioning element 1 can be inserted into a receiving section 8a of another tensioning element in such a way that both receiving sections align. A fixing means can be push through the aligned region and afterwards be inserted into the bone.

Between the aligned sections 10 and the receiving sections 8a, 8b there are provided docking points 12 where the hinges 2, 4 are provided as articulations. The hinges 2 and 4 are configured rotatable. The receiving section 8a is composed of two parts which are connected to each other via tow hinges 4.

The application of the tensioning element 1 for fixing a breaking point can take place as described in the following.

The angled sections 10 of the tensioning element 1 are pressed together from the outside, whereby the tensioning element 1 experiences a lateral contraction. Hereby, a longitudinal expansion of the tensioning element 1 occurs, i.e. the distance between the receiving sections 8a, 8b increases. Then two not shown fixing means, for example cortical screws, are pushed through so that with the increased distance between the receiving sections 8a, 8b the fixing means rest against the inner surfaces thereof. In this position, the fixing means are inserted into opposite fracture end of the bone. Due to the fixing means, after the removal of the external force, the tensioning element 1 is hold in the longitudinally extended shape, i.e. under a preload. The preload applies a continuous tensile force between the fixing means. The tensile force between the fixing means is used in the fixing device for the adaption of the fracture ends in a bone fracture.

FIG. 4 is a perspective view and FIG. 5 is a top view of a second embodiment of the tensioning element 1′ of the present invention with node joints.

The tensioning element 1′ is composed of a contoured body which is hollow when viewed from above and which comprises a circumferential wall 6′. The contoured body comprises two receiving sections 8a′, 8b′ which lie opposite to each other and which are curved outwardly. Further, the contoured body comprises two lateral flanks with angled sections 10′ which lie opposite to each other and which are slightly curved outward. Instead of the hinges 2, 4 of the first embodiment, there are formed node joints 14 at the docking points 12′. The node joints 14 are designed in such a way that these can either be deformed or pivoted.

FIG. 6 is a top view of a third embodiment of a tensioning element 1″ of the present invention having node joints 16. The tensioning element 1″ is composed of a contoured body which is hollow when viewed from above and which comprises a circumferential wall 6″. The contoured body comprises two receiving sections 8a″, 8b″ which lie opposite to each other and which are curved outward. Further, the contoured body comprises two lateral flanks with angled sections 10″ which lie opposite to each other and which are curved outward. In each of the both lateral flanks tow node joints 16 are formed in order to reduce the maximum stresses occurring in the angled sections 10″. Moreover, in each lateral flank a restriction bar 18 is formed between the node joints 16 so that the angled sections 10″ can be compressed solely for a predetermined level.

The tensioning element 1, 1′, 1″ is manufactured from biocompatible materials. Additionally, also a hybrid structure of the tensioning element 1, 1′, 1″ is possible in which the wall of the angled sections 8 consists of a material with the desired elastic property, and in which further sections of the tensioning element 1, 1′, 1″, as e.g. the receiving sections 6a, 6b, are formed of a rigid or absorbable material.

As a basic material for the tensioning element 1, 1′, 1″, for instance metals from the group: X42CrMo15, X100CrMo17, X2CrNiMnMoNNb21-16-5-3, X20Cr13, X15Cr13, X30Cr13, X46Cr13, X17CrNi16-2, X14CrMoS17, X30CrMoN15-1, X65CrMo 17-3, X55CrMo14, X90CrMoV18, X50CrMoV15, X 38CrMo V15, G-X 20CrMo13, X39CrMo17-1, X40CrMoVN16-2, X105CrMo17, X20CrNiMoS 13-1, X5CrNi18-0, X8CrNiS18-9, X2CrNi19-11, X2CrNi18-9, X10CrNi18-8, X5CrNiMo17-12-2, X2CrNiMo17-12-2, X2CrNiMoN25-7-4, X2CrNiMoN17-13-3, X2CrNiMo17-12-3, X2CrNiMo18-14-3, X2CrNiMo18-15-3; X 2 CrNiMo 18 14 3, X13CrMnMoN18-14-3, X2CrNiMoN22136, X2CrNiMnMoNbN21-9-4-3, X4CrNiMnMo21-9-4, X105CrCoMo18-2, X6CrNiTi18-10, X5CrNiCuNb16-4, X3CrNiCuTiNb12-9, X3CrNiCuTiNb12-9, X7CrNiAl17-7, CoCr2ONi15Mo, G-CoCr29Mo, CoCr20W15Ni, Co-20Cr-15W-10Ni, CoCr28MoNi, CoNi35Cr20Mo10, Ti1, Ti2, Ti3, Ti4, Ti-5Al-2,5Fe, Ti-5Al-2,5Sn, Ti-6Al-4V, Ti-6Al-4V ELI, Ti-3Al-2,5V (Gr9), 99,5Ti, Ti-12Mo-6Zr-2Fe, Ti-13,4Al-29Nb, Ti-13Nb-13Zr, Ti-15Al, Ti-15Mo, Ti-15Mo-5Zr-3Al, Ti-15Sn, Ti-15Zr-4Nb, Ti-15Zr-4Nb-4Ta, Ti-15Zr-4Nb-4Ta-0,2Pd, Ti-29Nb-13Ta-4,6Zr, Ti-30Nb-10Ta-5Zr, Ti-35,5Nb-1,5Ta-7,1Zr, Ti-35Zr-10Nb, Ti-45Nb, Ti-30Nb, Ti-30Ta, Ti-6Mn, Ti-5Zr-3Sn-5Mo-15Nb, Ti-3Al-8V-6Cr-4Zr-4Mo, Ti-6Al-2Nb-1Ta-0,8Mo, Ti-6Al-4Fe, Ti-6Al-4Nb, Ti-6Al-6Nb-1Ta, Ti-6Al-7Nb, Ti-6Al-4Zr-2Sn-2Mo, Ti-8,4Al-15,4Nb, Ti-8Al-7Nb, Ti-8Al-1Mo-1V, Ti-11Mo-6Zr-4Sn, may be considered.

Furthermore, polymers from the group: MBS, PMMI, MABS, CA, CTA, CAB, CAP, COC, PCT, PCTA, PCTG, EVA, EVAL, PTFE, ePTFE, PCTFE, PVDF, PVF, ETFE, ECTFE, FEP, PFA, LCP, PMMA, PMP, PHEMA, Polyamide 66, Polyamide 6, Polyamide 11, Polyamide 2, PAEK, PEEK, PB, PC, PPC, PETP, PBT, MDPE, LDPE, HDPE, UHMWPE, LLDPE, PI, PAI, PEI, PIB, POM, PPO, PPE, PPS, PP, PS, PSU, PESU, PVC, PVC-P, PVC-U, ABS, SAN, TPE-U, TPE-A, TPE-E, PVDC, PVA, SI, PDMS, EPM, EP, UF, MF, PF, PUR, UP, PEBA, PHB, PLA, PLLA, PDLA, PDLLA, PGL, PGLA, PGLLA, PGDLLA, PGL-co-poly TMC, PGL-co-PCL, PDS, PVAL, PCL, Poly-TMC, PUR (linear), NiTi Superelastic, NiTi Shape Memory, may be considered.

Furthermore, also ceramics from the group: Al₂O₃ (alumina oxide), Y-TZP (zirconium oxide ceramic), AMC (alumina matrix composite), HA (hydroxyl apatite), TCP (tricalcium phosphate), Ceravital (glass ceramic/Bioglas®), FZM/K (zirconium oxide, partially stabilized), TZP-A (zirconium oxide ceramic), ATZ (alumina-toughened zirconia), C799 (alumina oxide ceramic), Schott 8625 (transponder glass), may be considered.

Furthermore, also any combinations thereof may be considered.

Apart from the shown embodiments, the invention also allows for further design approaches.

Although the second and third embodiments do not comprise a corresponding tongue-and-groove structure of the both receiving sections, same can the configured in such a way in order to get expanded to a modular system as shown in DE 20 2011 109808 U1.

In a further alternative embodiment the tensioning element can comprise at least one node joint 16 at each lateral flank and one node joint 14 at each docking point.

Although the above-mentioned embodiments are configured symmetrically with respect to the longitudinal axis L, it is also possible to arrange solely two articulations positioned diametric opposite to each other when viewed the tensioning element from above.

In the above-mentioned embodiments in each embodiment solely one kind of articulations has been formed, the tensioning element, however, can be configured also in such a way that it comprises different kinds of articulations. For example, hinges may be used in the face sided receiving section 8a and node joints may be used in the opposite face sided receiving section 8b. Ever other combination is also configurable.

Although the above-mentioned lateral flank comprises one angled section, the lateral flank may be configured in such a way that it comprises more of these angled sections.

Moreover, it should be taken into account that the particular receiving sections can merge into the angled sections.

1.-9. (canceled)

10. A tensioning element for fixing fracture ends of bones in a bone fracture, wherein the tensioning element comprises:
a contoured body which is hollow when viewed from above and comprises a circumferential wall, comprising
two face sided receiving sections lying opposite to each other, for receiving fixing elements which can be pushed through the contoured body, and
two lateral flanks having angled sections,
the circumferential wall of the contoured body being resiliently deformable at least in sections, in a region of the face sided receiving sections and in a region of the lateral angled sections, and
wherein a defined elastic behavior of a clamping element can be impressed via the angled sections in such a way that a predetermined tension can be generated between the receiving sections,
and wherein the tensioning element further comprises at least one articulation for compensation and distribution of stress peaks, the at least one articulation being present in a region of the circumferential wall.

11. The tensioning element of claim 10, wherein the at least one articulation is deformable.

12. The tensioning element of claim 10, wherein the at least one articulation is rotatable.

13. The tensioning element of claim 10, wherein at least one articulation is positioned at docking points of the angled sections at the receiving sections.

14. The tensioning element of claim 11, wherein at least one articulation is positioned at docking points of the angled sections at the receiving sections.

15. The tensioning element of claim 12, wherein at least one articulation is positioned at docking points of the angled sections at the receiving sections.

16. The tensioning element of claim 10, wherein at least one articulation is integrated in the lateral flanks.

17. The tensioning element of claim 11, wherein at least one articulation is integrated in the lateral flanks.

18. The tensioning element of claim 12, wherein at least one articulation is integrated in the lateral flanks.

19. The tensioning element of claim 10, wherein at two wall sections opposing each other restriction bars facing each other are formed, their ends contacting each other when the tensioning element is deformed.

20. The tensioning element of claim 11, wherein at two wall sections opposing each other restriction bars facing each other are formed, their ends contacting each other when the tensioning element is deformed.

21. The tensioning element of claim 12, wherein at two wall sections opposing each other restriction bars facing each other are formed, their ends contacting each other when the tensioning element is deformed.

22. The tensioning element of claim 13, wherein at two wall sections opposing each other restriction bars facing each other are formed, their ends contacting each other when the tensioning element is deformed.

23. The tensioning element of claim 16, wherein at two wall sections opposing each other restriction bars facing each other are formed, their ends contacting each other when the tensioning element is deformed.

24. The tensioning element of claim 10, wherein the at least one articulation comprises a hinge.

25. The tensioning element of claim 10, wherein the at least one articulation comprises a ball joint.

26. The tensioning element of claim 10, wherein the at least one articulation comprises a node joint.
